SEQUENCE LISTING (60 sequences)

SEQ ID NO: 1 - DNA, 29 bp, Artificial Sequence
  made in laboratory
agatcggaagagcgtcgtgtagggaaaga

SEQ ID NO: 2 - DNA, 29 bp, Artificial Sequence
  made in laboratory
tctttccctacacgacgctcttccgatct

SEQ ID NO: 3 - DNA, 29 bp, Artificial Sequence
  made in laboratory
agatcggaagagcgtcgtgtagggaaaga

SEQ ID NO: 4 - DNA, 33 bp, Artificial Sequence
  made in laboratory
  misc_feature (30)..(33): n is a, c, g, or t
tctttccctacacgacgctcttccgatctnnnn

SEQ ID NO: 5 - DNA, 33 bp, Artificial Sequence
  made in laboratory
  misc_feature (30)..(32): n is a, c, g, or t
tctttccctacacgacgctcttccgatctnnna

SEQ ID NO: 6 - DNA, 33 bp, Artificial Sequence
  made in laboratory
  misc_feature (30)..(31): n is a, c, g, or t
  misc_feature (33)..(33): n is a, c, g, or t
tctttccctacacgacgctcttccgatctnnan

SEQ ID NO: 7 - DNA, 33 bp, Artificial Sequence
  made in laboratory
  misc_feature (30)..(30): n is a, c, g, or t
  misc_feature (32)..(33): n is a, c, g, or t
tctttccctacacgacgctcttccgatctnann

SEQ ID NO: 8 - DNA, 33 bp, Artificial Sequence
  made in laboratory
  misc_feature (31)..(33): n is a, c, g, or t
tctttccctacacgacgctcttccgatctannn

SEQ ID NO: 9 - DNA, 43 bp, Artificial Sequence
  made in laboratory
  misc_feature (27)..(32): n is a, c, g, or t
agttcagacgtgtgctcttccgatctnnnnnnagatcggaagt

SEQ ID NO: 10 - DNA, 10 bp, Artificial Sequence
  made in laboratory
tctagccttc

SEQ ID NO: 11 - DNA, 60 bp, Artificial Sequence
  made in laboratory
aatgatacggcgaccaccgagatctacacacactttccctacacgacgctcttccgatct

SEQ ID NO: 12 - DNA, 60 bp, Artificial Sequence
  made in laboratory
caagcagaagacggcatacgagatgtgactggagagttcagacgtgtgctcttccgatct

SEQ ID NO: 13 - DNA, 8 bp, Artificial Sequence
  made in laboratory
cgaccatt

SEQ ID NO: 14 - DNA, 8 bp, Artificial Sequence
  made in laboratory
gatagcga

SEQ ID NO: 15 - DNA, 8 bp, Artificial Sequence
  made in laboratory
aatggacg

SEQ ID NO: 16 - DNA, 8 bp, Artificial Sequence
  made in laboratory
cgctagta

SEQ ID NO: 17 - DNA, 8 bp, Artificial Sequence
  made in laboratory
tctctagg

SEQ ID NO: 18 - DNA, 8 bp, Artificial Sequence
  made in laboratory
acattgcg

SEQ ID NO: 19 - DNA, 8 bp, Artificial Sequence
  made in laboratory
tgaggtgt

SEQ ID NO: 20 - DNA, 8 bp, Artificial Sequence
  made in laboratory
aatgcctc

SEQ ID NO: 21 - DNA, 8 bp, Artificial Sequence
  made in laboratory
ctggagta

SEQ ID NO: 22 - DNA, 8 bp, Artificial Sequence
  made in laboratory
gtatgctg

SEQ ID NO: 23 - DNA, 8 bp, Artificial Sequence
  made in laboratory
tggagagt

SEQ ID NO: 24 - DNA, 8 bp, Artificial Sequence
  made in laboratory
cgatagag

SEQ ID NO: 25 - DNA, 8 bp, Artificial Sequence
  made in laboratory
ctcattgc

SEQ ID NO: 26 - DNA, 8 bp, Artificial Sequence
  made in laboratory
accagctt

SEQ ID NO: 27 - DNA, 8 bp, Artificial Sequence
  made in laboratory
gaatcgtg

SEQ ID NO: 28 - DNA, 8 bp, Artificial Sequence
  made in laboratory
aggcttct

SEQ ID NO: 29 - DNA, 8 bp, Artificial Sequence
  made in laboratory
aatggtcg

SEQ ID NO: 30 - DNA, 8 bp, Artificial Sequence
  made in laboratory
tcgctatc

SEQ ID NO: 31 - DNA, 8 bp, Artificial Sequence
  made in laboratory
cgtccatt

SEQ ID NO: 32 - DNA, 8 bp, Artificial Sequence
  made in laboratory
tactagcg

SEQ ID NO: 33 - DNA, 8 bp, Artificial Sequence
  made in laboratory
cctagaga

SEQ ID NO: 34 - DNA, 8 bp, Artificial Sequence
  made in laboratory
cgcaatgt

SEQ ID NO: 35 - DNA, 8 bp, Artificial Sequence
  made in laboratory
acacctca

SEQ ID NO: 36 - DNA, 8 bp, Artificial Sequence
  made in laboratory
gaggcatt

SEQ ID NO: 37 - DNA, 8 bp, Artificial Sequence
  made in laboratory
tactccag

SEQ ID NO: 38 - DNA, 8 bp, Artificial Sequence
  made in laboratory
cagcatac

SEQ ID NO: 39 - DNA, 8 bp, Artificial Sequence
  made in laboratory
actctcca

SEQ ID NO: 40 - DNA, 8 bp, Artificial Sequence
  made in laboratory
ctctatcg

SEQ ID NO: 41 - DNA, 8 bp, Artificial Sequence
  made in laboratory
gcaatgag

SEQ ID NO: 42 - DNA, 8 bp, Artificial Sequence
  made in laboratory
aagctggt

SEQ ID NO: 43 - DNA, 8 bp, Artificial Sequence
  made in laboratory
cacgattc

SEQ ID NO: 44 - DNA, 8 bp, Artificial Sequence
  made in laboratory
agaagcct

SEQ ID NO: 45 - DNA, 8 bp, Artificial Sequence
  made in laboratory
gcctatca

SEQ ID NO: 46 - DNA, 8 bp, Artificial Sequence
  made in laboratory
cttggatg

SEQ ID NO: 47 - DNA, 8 bp, Artificial Sequence
  made in laboratory
agtctcac

SEQ ID NO: 48 - DNA, 8 bp, Artificial Sequence
  made in laboratory
ctcatcag

SEQ ID NO: 49 - DNA, 8 bp, Artificial Sequence
  made in laboratory
tgtaccgt

SEQ ID NO: 50 - DNA, 8 bp, Artificial Sequence
  made in laboratory
aagtcgag

SEQ ID NO: 51 - DNA, 8 bp, Artificial Sequence
  made in laboratory
cacgttgt

SEQ ID NO: 52 - DNA, 8 bp, Artificial Sequence
  made in laboratory
tcacagca

SEQ ID NO: 53 - DNA, 8 bp, Artificial Sequence
  made in laboratory
ctacttgg

SEQ ID NO: 54 - DNA, 8 bp, Artificial Sequence
  made in laboratory
cctcagtt

SEQ ID NO: 55 - DNA, 8 bp, Artificial Sequence
  made in laboratory
tcctacct

SEQ ID NO: 56 - DNA, 8 bp, Artificial Sequence
  made in laboratory
atggcgaa

SEQ ID NO: 57 - DNA, 8 bp, Artificial Sequence
  made in laboratory
cttacctg

SEQ ID NO: 58 - DNA, 8 bp, Artificial Sequence
  made in laboratory
ctcgatac

SEQ ID NO: 59 - DNA, 8 bp, Artificial Sequence
  made in laboratory
tccgtgaa

SEQ ID NO: 60 - DNA, 8 bp, Artificial Sequence
  made in laboratory
tagagctc

(19) United States
(12) Patent Application Publication
(10) Pub. No.: US 2025/0297406 A1
(43) Pub. Date: Sep. 25, 2025

(54) ADAPTERS, ADAPTER LIGATION REAGENT, KIT, METHOD FOR CONSTRUCTING DNA LIBRARY AND METHOD FOR SEQUENCING GENE

(71) Applicants: CHENGDU BOE OPTOELECTRONICS TECHNOLOGY CO., LTD., Sichuan (CN); BOE TECHNOLOGY GROUP CO., LTD., Beijing (CN)

(72) Inventors: Bangquan YE, Beijing (CN); Dandan CHEN, Beijing (CN)

(73) Assignees: CHENGDU BOE OPTOELECTRONICS TECHNOLOGY CO., LTD., Sichuan (CN); BOE TECHNOLOGY GROUP CO., LTD., Beijing (CN)

(21) Appl. No.: 18/274,965

(22) PCT Filed: Apr. 18, 2022
(86) PCT No.: PCT/CN2022/087490

§ 371 Date: Jul. 28, 2023

(51) Int. Cl. C40B 50/06; C12N 15/10; C12N 15/11; C12Q 1/6806

(52) U.S. Cl. CPC C40B 50/06; C12N 15/10; C12N 15/11; C12Q 1/6806

(57) ABSTRACT

Adapters are provided. An adapter includes at least one first sub-adapter. Each first sub-adapter includes: a first nucleotide single strand and a second nucleotide single strand, the first nucleotide single strand being complementarily paired with the second nucleotide single strand; and a first nucleotide single strand segment, the first nucleotide single strand segment being ligated to an end of the first nucleotide single strand or an end of the second nucleotide single strand. The first nucleotide single strand segment includes at least one random base and at least one adenine (A) base. Each random base is any one of an A base, a cytosine (C) base, a guanine (G) base and a thymine (T) base.

### CROSS-REFERENCE TO RELATED APPLICATION

The application is a national phase entry under 35 USC 371 of International Patent Application No. PCT/CN2022/087490 filed on Apr. 18, 2022, which is incorporated herein by reference in its entirety.

### TECHNICAL FIELD

The present disclosure relates to the field of biotechnologies, and in particular, to adapters, an adapter ligation reagent, a kit, a method for constructing a library and a method for sequencing a gene.

### BACKGROUND

High-throughput sequencing is also referred to as massively parallel sequencing or next generation sequencing. High throughput sequencing is capable of sequencing a plurality of target regions of a single sample or a plurality of samples at a time, and applications thereof in clinical practice including pharmacogenomics, genetic disease research and screening, tumor mutation gene detection and clinical microbial detection are gaining increasing attention. Next generation sequencing technologies, which are currently most widely used sequencing technologies, have advantages of high sequencing depth, large throughput, high accuracy and good sensitivity.

### SUMMARY

In one aspect, an adapter is provided. The adapter includes at least one first sub-adapter. Each first sub-adapter includes a first nucleotide single strand, a second nucleotide single strand and a first nucleotide single strand segment. The first nucleotide single strand is complementarily paired with the second nucleotide single strand. The first nucleotide single strand segment is ligated to an end of the first nucleotide single strand or an end of the second nucleotide single strand. The first nucleotide single strand segment includes at least one random base and at least one adenine (A) base. Each random base is any one of an A base, a cytosine (C) base, a guanine (G) base and a thymine (T) base.

In some embodiments, the first nucleotide single strand segment includes a plurality of random bases and at least one A base, and the plurality of random bases are arranged consecutively; or the first nucleotide single strand segment includes a plurality of A bases and at least one random base, and the plurality of A bases are arranged consecutively.

In some embodiments, the first nucleotide single strand segment includes a plurality of random bases and at least one A base, one or more A bases of the at least one A base are disposed between two random bases of the plurality of random bases; or the first nucleotide single strand segment includes a plurality of A bases and at least one random base, and one or more random bases of the at least one random base are disposed between two A bases of the plurality of A bases.

In some embodiments, the first nucleotide single strand segment includes three random bases and one A base.

In some embodiments, the adapter includes a plurality of first sub-adapters. Among the plurality of first sub-adapters, at least two first sub-adapters are different in that random bases and A bases of respective first nucleotide single strand segments are arranged in different orders.

In some embodiments, the adapter includes four first sub-adapters. Four first nucleotide single strand segments of the four first sub-adapters are different in that random bases and A bases of respective first nucleotide single strand segments are arranged in different orders.

In another aspect, an adapter is provided. The adapter includes at least one second sub-adapter. Each second sub-adapter includes a third nucleotide single strand, a fourth nucleotide single strand and a second nucleotide single strand segment. The third nucleotide single strand is complementarily paired with the fourth nucleotide single strand. The second nucleotide single strand segment is ligated to an end of the third nucleotide single strand or an end of the fourth nucleotide single strand. The second nucleotide single strand segment includes at least one random base. Each random base is any one of an A base, a C base, a G base and a T base.

In some embodiments, the second nucleotide single stranded segment includes four random bases.

In yet another aspect, an adapter ligation reagent is provided. The adapter ligation reagent includes the adapter as described above.

In some embodiments, the adapter ligation reagent further includes at least one second sub-adapter. Each second sub-adapter includes a third nucleotide single strand, a fourth nucleotide single strand and a second nucleotide single strand segment. The third nucleotide single strand is complementarily paired with the fourth nucleotide single strand. The second nucleotide single strand segment is ligated to an end of the third nucleotide single strand or an end of the fourth nucleotide single strand. The second nucleotide single strand segment includes at least one random base, each random base being any one of an A base, a C base, a G base and a T base.

In some embodiments, the adapter ligation reagent further includes a third sub-adapter. The third sub-adapter includes a fifth nucleotide single strand, a sixth nucleotide single strand and at least one unique molecular identifier (UMI). The fifth nucleotide single strand is complementarily paired with the sixth nucleotide single strand. each UMI is located on the fifth nucleotide single strand or the sixth nucleotide single strand.

In yet another aspect, a kit is provided. The kit includes the adapter ligation regent as described above.

In some embodiments, the adapter ligation reagent further includes a third sub-adapter. The third sub-adapter includes a fifth nucleotide single strand, a sixth nucleotide single strand and at least one unique molecular identifier (UMI). The fifth nucleotide single strand is complementarily paired with the sixth nucleotide single strand. Each UMI is located on the fifth nucleotide single strand or the sixth nucleotide single strand.

In some embodiments, the UMI includes at least one random base. Each random base is any one of an adenine (A) base, a cytosine (C) base, a guanine (G) base and a thymine (T) base.

In some embodiments, the at least one random base includes at least six random bases.

In some embodiments, the at least one UMI includes one UMI. The UMI is located on the fifth nucleotide single strand.

In some embodiments, the fifth nucleotide single strand is a forward strand, and the sixth nucleotide single strand is a reverse strand. The fifth nucleotide single strand includes a sequencing primer sequence and an amplification primer sequence. The UMI located on the fifth nucleotide single strand is located between the sequencing primer sequence and the amplification primer sequence. The sequencing primer sequence is combined with bases of the sixth nucleotide single strand through complementary base pairing.

In yet another aspect, a method for constructing a deoxyribonucleic acid (DNA) library is provided. The method includes: obtaining degraded DNA; melting the degraded DNA to form single-stranded DNA; performing treatment, by using the adapter ligation reagent as described above, to make the adapter, at least one first sub-adapter and the at least one second sub-adapter of the adapter ligation reagent as described above react with the single-stranded DNA to obtain adapter ligation products; and purifying and enriching the adapter ligation products to obtain the DNA library.

In some embodiments, the adapter ligation reagent further includes at least one third sub-adapter. The third sub-adapter includes a fifth nucleotide single strand, a sixth nucleotide single strand and at least one unique molecular identifier (UMI). The fifth nucleotide single strand is complementarily paired with the sixth nucleotide single strand. Each UMI is located on the fifth nucleotide single strand or the sixth nucleotide single strand. The method includes: performing treatment, by using the adapter ligation reagent, to make the at least one first sub-adapter, the at least one second sub-adapter and the third sub-adapter of the adapter ligation reagent react with the single-stranded DNA to obtain the adapter ligation products.

In yet another aspect, a method for sequencing a gene is provided. The method includes performing gene sequencing on DNA obtained by using the method for constructing the DNA library as described above.

### BRIEF DESCRIPTION OF THE DRAWINGS

In order to describe technical solutions in the present disclosure more clearly, accompanying drawings to be used in some embodiments of the present disclosure will be introduced briefly below. However, the accompanying drawings to be described below are merely accompanying drawings of some embodiments of the present disclosure, and a person having ordinary skill in the art can obtain other drawings according to these accompanying drawings. In addition, the accompanying drawings in the following description may be regarded as schematic diagrams, but are not limitations on an actual size of a product, an actual process of a method and an actual timing of a signal involved in the embodiments of the present disclosure.

FIG. 1 is a structural diagram of a first sub-adapter, in accordance with some embodiments;

FIGS. 2A to 2D are structural diagrams of some other first sub-adapters, in accordance with some embodiments;

FIGS. 3A and 3B are structural diagrams of yet some other first sub-adapters, in accordance with some embodiments;

FIGS. 4A and 4B are structural diagrams of second sub-adapters, in accordance with some embodiments;

FIG. 5 is a flowchart of a sequencing method, in accordance with some embodiments;

FIG. 6 is a flowchart for constructing a library, in accordance with some embodiments; and

FIG. 7 is a flowchart for constructing another library, in accordance with some embodiments.

### DETAILED DESCRIPTION

Technical solutions in some embodiments of the present disclosure will be described clearly and completely below with reference to the accompanying drawings. However, the described embodiments are merely some but not all embodiments of the present disclosure. All other embodiments obtained by a person of ordinary skill in the art based on the embodiments of the present disclosure shall be included in the protection scope of the present disclosure.

Unless the context requires otherwise, throughout the description and the claims, the term “comprise” and other forms thereof such as the third-person singular form “comprises” and the present participle form “comprising” are construed as an open and inclusive meaning, i.e., “including, but not limited to”. In the description of the specification, the terms such as “one embodiment”, “some embodiments”, “exemplary embodiments”, “example”, “specific example” or “some examples” are intended to indicate that specific features, structures, materials or characteristics related to the embodiment(s) or example(s) are included in at least one embodiment or example of the present disclosure. Schematic representation of the above terms does not necessarily refer to the same embodiment(s) or example(s). In addition, the specific features, structures, materials or characteristics may be included in any one or more embodiments or examples in any suitable manner.

Hereinafter, the terms such as “first” and “second” are used for descriptive purposes only, but are not to be construed as indicating or implying the relative importance or implicitly indicating the number of indicated technical features. Thus, the features defined with “first” and “second” may explicitly or implicitly include one or more of the features. In the description of the embodiments of the present disclosure, the term “a plurality of/the plurality of” means two or more unless otherwise specified.

The phrase “at least one of A, B and C” has a same meaning as the phrase “at least one of A, B or C”, and they both include the following combinations of A, B and C: only A, only B, only C, a combination of A and B, a combination of A and C, a combination of B and C, and a combination of A, B and C.

The phrase “A and/or B” includes the following three combinations: only A, only B, and a combination of A and B.

The phrase “applicable to” or “configured to” as used herein indicates an open and inclusive expression, which does not exclude devices that are applicable to or configured to perform additional tasks or steps.

In addition, the use of the phrase “based on” is meant to be open and inclusive, since a process, step, calculation or other action that is “based on” one or more of the stated conditions or values may, in practice, be based on additional conditions or values exceeding those stated.

Terms such as “about”, “substantially” or “approximately” as used herein include a stated value and an average value within an acceptable range of deviation of a particular value. The acceptable range of deviation is determined by a person of ordinary skill in the art in view of the measurement in question and the error associated with the measurement of a particular quantity (i.e., the limitations of the measurement system).

As used herein, the term “DNA” is an abbreviation for deoxyribonucleic acid. DNA is a carrier of genetic information existing in biological cells, and mainly used for guiding synthesis of ribonucleic acid (RNA) and proteins in a body. The DNA is a macromolecular polymer composed of deoxynucleotides. A deoxynucleotide is composed of a phosphate, a deoxyribose and a base. There are four main kinds of bases, i.e., adenine (A), guanine (G), cytosine (C) and thymine (T).

As used herein, the term “RNA” is an abbreviation for ribonucleic acid. The RNA is a carrier of genetic information existing in biological cells and some viruses and viroids, and mainly used for guiding synthesis of proteins in the body. The RNA is a macromolecular polymer composed of ribonucleotides. A ribonucleotide is composed of a phosphate, a ribose and a base. There are four main kinds of bases, i.e., adenine (A), guanine (G), cytosine (C) and uracil (U).

Conventional DNA library construction is usually performed on double-stranded DNA, and includes the following steps. In a step 1, DNA is fragmented; in a step 2, end-repair and adenine (A) addition are performed; in a step 3, ligation of double-stranded adapters is performed; and in a step 4, ligation products are amplified and enriched to form a library. The double-stranded adapters are only applicable to double-stranded DNA. In some severely degraded DNA samples, DNA usually exists in both a single-stranded form and a double-stranded form. In addition, a portion of double-stranded DNA has a problem such as a broken strand or intermittent deletion. Such a DNA sample may be an extracellular circulating DNA sample, or a formalin-fixed and paraffin-embedded biological tissue sample, a forensic sample, a DNA sample extracted from a paleontological fossil, etc. For single-stranded DNA or double-stranded DNA with breakage or intermittent deletion, it is prone to loss of single-stranded DNA if the conventional double-stranded library construction strategy is adopted. Consequently, a problem such as false negative or low sensitivity is caused in a subsequent detection. Especially in the field of DNA methylation sequencing, after DNA is treated by sulfite, DNA templates are broken, and a large amount of single-stranded DNA are formed. Thus, if the conventional double-stranded library construction method is adopted, massive loss of the single-stranded DNA seriously affects sensitivity of subsequent detection of CpG sites. A single-strand library construction method, in which adapters are perfectly applicable to single-strand DNA, may fully ensure that the single-strand DNA effectively form a library for subsequent experiments such as sequencing, which avoids a loss of a sample. Therefore, single-stranded DNA library construction is very suitable for the field of circulating tumor DNA (ctDNA) methylation sequencing.

Single-strand library building technologies in the current market mainly has the following two technical approaches. A first technical approach is represented by Accel-NGS Methyl-seq technique from Swift. That is, a nucleotide sequence including an illumina universal sequence is firstly ligated to a 3′ end of single-stranded DNA by means of a single-stranded ligase (such as a circligase II) which is extremely expensive, and then amplification is performed to form double strands by means of a complementary primer of the universal sequence, and then a double-stranded adapter is added conventionally to form a complete product available for sequencing to perform sequencing. The technique is extremely costly due to the use of the single-stranded ligase, and ligation efficiency is low in a case of a large input amount of DNA; and in addition, a serious ligation bias problem exists in a DNA sample treated by sulfite. A second technical approach is the QIAseq Methyl Library Kit from Qiagen. A principle of the kit is that, a random sequence of 8 base pairs (bp) is designed as a primer and amplified to form double strands, and then a double-stranded adapter is used for ligation. This method has a certain bias in polymerase chain reaction (PCR) amplification, which results in inefficient library construction. An additional problem existing in both of the above two library construction approaches is an absence of molecular tags. Thus, the two approaches are incapable of redundancy removal and correcting errors introduced by PCR amplification and sequencing.

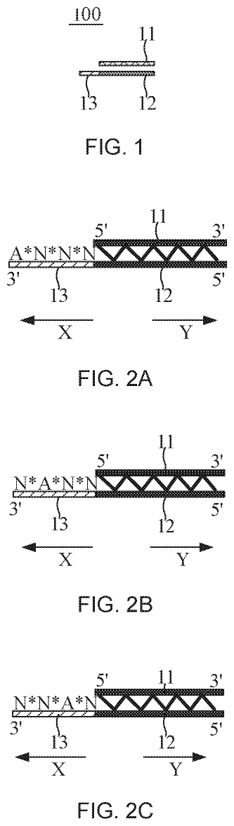

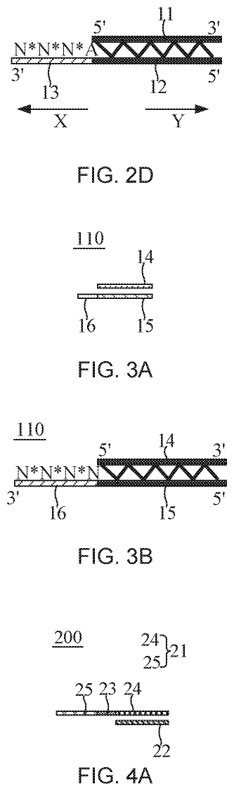

In view of the above technical problems, as shown in FIG. 1, some embodiments of the present disclosure provide adapters. The adapters may be named as first adapters. The first adapters include at least one first sub-adapter 100. Each first sub-adapter 100 includes a first nucleotide single strand 11, a second nucleotide single strand 12 and a first nucleotide single strand segment 13. The first nucleotide single strand 11 is complementarily paired with the second nucleotide single strand 12. The first nucleotide single strand segment 13 is ligated to an end of the first nucleotide single strand 11 or an end of the second nucleotide single strand 12. As shown in FIGS. 2A to 2D, the first nucleotide single strand segment 13 includes at least one random base and at least one A base. Each random base is any one of an A base, a C base, a G base and a T base. A random base may be represented by N.

Since a percentage of C bases in human genome DNA is about 22.5%, and a percentage of unmethylated C bases therein is about 16.5%. After the DNA is treated by sulfite, the unmethylated C bases are converted into U bases, which causes that percentages of bases in a sequence are changed. It is expected that a percentage of C bases is 6%, a percentage of U bases and T bases is 44%, and percentages of G bases and A bases remain unchanged. Therefore, for a sulfite-treated sequence, there is base imbalance, and a percentage of U bases and T bases is large. A single-stranded ligation adapter in prior art carries out a ligation reaction by using four to eight N bases at an end thereof. However, percentages of four kinds of bases, i.e., A bases, G bases, C bases and T bases, in the N bases are each 25%. Thus, a success rate of complementary pairing between conventional adapters with N bases and sulfite-treated DNA is low, which indirectly causes a reduction of an amount of ligation products, i.e., low ligation efficiency, in the present of a T4 DNA ligase. For an adapter (i.e., an adapter including a first sub-adapter 100) in embodiments of the present disclosure, a percentage of A bases in a first nucleotide single strand section 13 is increased to a range of 40% to 50%. In this way, a success rate of complementary pairing between the adapters and the sulfite treated single-stranded DNA is improved, which solves the problem of low ligation efficiency.

In some embodiments, as shown in FIG. 1, the first nucleotide single strand segment 13 is ligated to the end of the second nucleotide single strand 12.

It will be noted that, the first nucleotide single strand segment 13 may also be ligated to the end of the first nucleotide single strand 11. Explanation in embodiments of the present disclosure is made by taking an example where the first nucleotide single strand segment 13 is ligated to the end of the second nucleotide single strand 12. For example, as shown in FIGS. 2A to 2D, the first nucleotide single strand segment 13 is ligated to a 3′ end of the second nucleotide single strand 12.

In some embodiments, the first nucleotide single strand segment 13 includes a plurality of random bases and at least one A base, and the plurality of random bases are arranged consecutively.

For example, there are the plurality of random base and one A base. In this case, the plurality random bases are arranged consecutively. In this case, the A base may be located on a side of the plurality of random bases (for example, a direction from a 5′ to the 3′ end of the second nucleotide single strand 12 is referred to as a first direction X, and a direction from the 5′ end to the 3′ end of the second nucleotide single strand 12 is referred to as a second direction Y, and the A base may be located on a side of the plurality of random bases facing the first direction or the second direction).

For example, as shown in FIGS. 2A to 2D, the first nucleotide single strand segment 13 includes three random bases and one A base, and the A base is located on a side of the three random bases. As shown in FIG. 2A, the A base is located on a side of the three random bases facing the first direction X. As shown in FIG. 2D, the A base is located on a side of the three random bases facing the second direction Y.

For example, there are the plurality of random bases and a plurality of A bases. In this case, the plurality of random bases and the plurality of A bases are both arranged continuously. In this case, the plurality of A bases may be located on a side of the plurality of the random bases (for example, the direction from the 5′ to the 3′ end of the second nucleotide single strand 12 is referred to as the first direction X, the direction from the 5′ end to the 3′ end of the second nucleotide single strand 12 is referred to as the second direction Y, and the plurality of A bases may be located on a side of the plurality of the random bases facing the first direction X or the second direction Y).

In some other embodiments, the first nucleotide single stranded segment includes a plurality of A bases and at least one random base, and the plurality of A bases are arranged consecutively.

For example, there are the plurality of A bases and one random base. In this case, the random base may be located on a side of the plurality of bases (for example, the direction from the 5′ to the 3′ end of the second nucleotide single strand 12 is referred to as the first direction X, the direction from the 5′ end to the 3′ end of the second nucleotide single strand 12 is referred to as the second direction Y, and the random base may be located on a side of the plurality of A bases facing the first direction or the second direction), or the random base may be located between any two A bases.

In yet some other embodiments, the first nucleotide single stranded segment 13 includes a plurality of random bases and at least one A base, and one or more A bases of the at least one A base are disposed between two random bases of the plurality of random bases.

For example, there are the plurality of random bases and a plurality of A bases. The plurality of A bases may be located in any at least two random bases arranged at an interval. For example, the plurality of A bases are located between any two random bases arranged at an interval, and the plurality of A bases are located between any three random bases arranged at an intervals. Alternatively, for both the plurality of random bases and the plurality of A bases, at least two bases thereof are arranged at an interval, there may be one or more A bases between two random bases arranged at an interval, and there may be one or more random bases between two A bases arranged at an interval. Embodiments of the present disclosure are not limited thereto.

For example, as shown in FIGS. 2B and 2C, the first nucleotide single strand segment includes three random bases and one A base. The A base is located between any two random bases. As shown in FIG. 2B, in the first direction X, the A base is located between an N base at a second location and an N base at a fourth location.

As shown in FIG. 2C, in the first direction X, the A base is located between an N base at a first location and an N base at a third location.

In yet some other embodiments, the first nucleotide single stranded segment 13 includes a plurality of A bases and at least one random base, and one or more random bases of the at least one random base are disposed between two A bases of the plurality of A bases.

For example, the first nucleotide single strand segment 13 includes one random base and three A bases. In this case, the one random base is located between any two A bases.

As can be seen from the above, there are three random bases and one A base in the first sub-adapter 100 as shown in FIGS. 2A to 2D, and there are two cases for the first sub-adapter 100. In a first case, as shown in FIGS. 2A and 2D, the A base may be located on a side of the three random bases (for example, the direction from the 5′ to the 3′ end of the second nucleotide single strand 12 is referred to as the first direction X, the direction from the 5′ end to the 3′ end of the second nucleotide single strand 12 is referred to as the second direction Y, and the A base may be located on a side of the three random bases facing the first direction or the second direction). In a second case, as shown in FIGS. 2B and 2C, the A base is located between any two random bases. In this way, the percentage of A bases in the first nucleotide single strand section 13 is increased, which increases the success rate of complementary pairing with the single-stranded DNA, and then improves the ligation efficiency.

In some embodiments, the adapters include a plurality of first sub-adapters 100. Among the plurality of first sub-adapters 100, at least two first sub-adapters 100 are different in that random bases and A bases of respective first nucleotide single strand segments 13 are arranged in different orders. For example, random bases and bases A of first nucleotide single strand segments 13 of two first sub-adapters 100 may be arranged in any two of arrangement orders as shown in FIGS. 2A to 2D, respectively.

In some embodiments, the adapters include four first sub-adapters 100. The four first sub-adapters 100 are different in that random bases and A bases of respective first nucleotide single strand segments 13 are arranged in different orders. For example, random bases and A bases of first nucleotide single strand segments 13 of the four first sub-adapters 100 may be arranged in the orders shown in FIGS. 2A to 2D, respectively.

In some embodiments, as shown in FIG. 3A, the first adapters further include at least one second sub-adapter 110. Each second sub-adapter 110 includes a third nucleotide single strand 14, a fourth nucleotide single strand 15 and a second nucleotide single strand segment 16. The third nucleotide single strand 14 is complementarily paired with the fourth nucleotide single strand 15. The second nucleotide single strand segment 16 is ligated to an end of the third nucleotide single strand 14 or an end of the fourth nucleotide single strand 15. The second nucleotide single strand segment 16 includes at least one random base. Each random base is any one of an A base, a C base, a G base and a T base. A random base may be represented by N.

In some embodiments, as shown in FIG. 3A, the second nucleotide single strand segment 16 is ligated to an end of the fourth nucleotide single strand 15. As shown in FIG. 3B, the second nucleotide single strand segment 16 is ligated to a 3′ end of the fourth nucleotide single strand 15.

It will be noted that, the second nucleotide single strand segment 16 may be ligated to an end of the third nucleotide single strand 14. Explanation in embodiments of the present disclosure is made by taking an example where the second nucleotide single strand segment 16 is ligated to the end of the third nucleotide single strand 14.

In some embodiments, as shown in FIG. 3B, there are four random bases, each random base is any one of an A base, a C base, a G base and a T base, and a random base may be represented by N. In this case, there are 4⁴ cases for the second single strand segment 16. It can be seen that, as the random bases and kinds of specific bases as options become more numerous, kinds of the second nucleotide single strand segment 16 becomes more numerous.

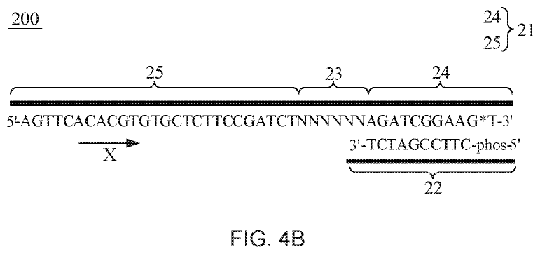

As shown in FIG. 4A, embodiments of the present disclosure provide another adapter. The another adapter may be named as a second adapter. The second adapter includes a third sub-adapter 200. The third sub-adapter 200 includes a fifth nucleotide single strand 21, a sixth nucleotide single strand 22 and at least one unique molecular identifier (UMI) 23. The fifth nucleotide single strand 21 is complementarily paired with the sixth nucleotide single strand 22. Each UMI 23 is located on the fifth nucleotide single strand 21 or the sixth nucleotide single strand 22.

In some embodiments, the UMI 23 includes at least one random base. Each random base is any one of an A base, a C base, a G base and a T base. A random base may be represented by N. Different bases are selected as random bases, which allows a use in labeling different DNA molecules.

For example, considering an example where there is one random base in a single UMI 23, an N of the UMI 23 may be any one of the four kinds of bases. In this case, depending on different N bases of UMIs 23, 4 kinds of UMIs 23 may be obtained. The 4 kinds of UMIs 23 may be made into 4² (i.e., 16) adapters (a single DNA molecule is ligated with two adapters), so that 4² (i.e., 16) different DNA molecules may be labeled, and then detections for the 4² (i.e., 16) different DNA molecules may be achieved.

Considering an example where there are 3 random bases in a single UMI 23, each N of the UMI 23 may be any one of the four kinds of bases. In this case, there may be 4³ (i.e., 64) combinations depending on the 3 N bases of the UMI 23, so that 4³ (i.e., 64) kinds of UMIs 23 may be obtained. The 64 kinds of UMIs 23 may be made into 64² (i.e., 4096) adapters (a single DNA molecule is ligated with two adapters), so that 64² (i.e., 4096) different DNA molecules may be labeled, and then detections for the 64² (i.e., 4096) different DNA molecules may be achieved.

Considering an example where there are 6 random bases in a single UMI 23, each N in the UMI 23 may be any one of the four kinds of bases. In this case, there may be 4⁶ (4096) combinations depending on the 6 N bases of the UMI, so that 4⁶ (4096) kinds of UMIs may be obtained, and the 4096 kinds of UMIs 23 may be made into 4096² (16777216) adapters (a single DNA molecule is ligated with two adapters), so that 4096² (16777216) different DNA molecules may be labeled, and then detections for the 4096² (16777216) different DNA molecules are achieved.

It can be seen that, as random bases become more numerous, kinds of the UMIs 23 becomes more numerous, and then more DNA molecules may be labeled by the UMIs 23.

The UMI 23 included in the third sub-adapter 200 is used for correcting errors introduced by PCR amplification and sequencing, so as to avoid noise mutation.

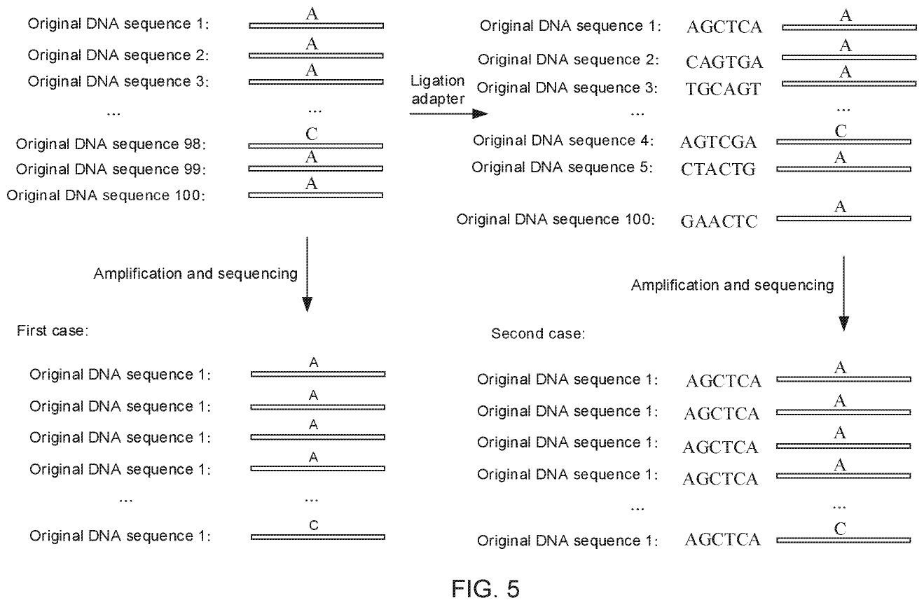

For example, as shown in FIG. 5, 100 original DNA fragments (i.e., sequences from different cells) with same starting and ending positions are respectively identified as original DNA sequence 1, original DNA sequence 2, original DNA sequence 3 . . . original DNA sequence 99 and original DNA sequence 100. The original DNA sequence 98 is a mutated sequence, where an A base therein is mutated to a C base. That is, a mutation frequency is 1%. The original DNA fragments are respectively ligated with UMI adapters to obtain sequences corresponding to the original DNA sequence 1 to the original DNA sequence 100. The obtained sequences are still marked as original DNA sequence 1, original DNA sequence 2, original DNA sequences 3 . . . original DNA sequence 99 and original DNA sequence 100. PCR amplification and enrichment are performed on the 100 original DNA sequences ligated with the UMIs adapters to obtain a DNA library. The DNA library includes 100 original DNA sequences 1 ligated with UMI adapters.

In the library construction process, the PCR amplification and enrichment here means that, the original DNA sequences are used as templates to perform PCR amplification so as to copy completely same original DNA sequences. However, during amplification, an amplification error occurs due to factors such as enzyme activity. As shown in a first case in FIG. 5, each original DNA sequence is not ligated with a UMI adapter. In this case, the amplification error cannot be eliminated, and may be mistaken for a true mutation, which results in a detection result of false positive. In a case where the original DNA sequence is amplified and copied after being ligated with a UMI, and an amplification error occurs, as shown in a second case in FIG. 5 where each original DNA sequence is ligated with a UMI adapter, it may be possible to determine the amplification error rather than a true mutation according to exactly same sequences of UMI adapters.

It can be seen that, by using UMIs 23 in third sub-adapters 200, it may be possible to label different original DNA fragments and eliminate noisy mutation(s) introduced by PCR amplification or sequencing, thereby improving the detection accuracy.

In some embodiments, there are at least 6 random bases.

For example, there are 6 to 8 random bases. There may be 6, 7 or 8 random bases. In this way, it may be possible to avoid an occupation of a subsequent sequencing data volume caused by an excessive number of random bases under a premise of ensuring a detection fault tolerance. As shown in FIG. 4B, illustration in embodiments of the present disclosure is made by taking an example where there are 6 random bases. Since a random base may be any one of an A base, a C base, a G base and a T base, there exists 4⁶ combinations which are sufficient to distinguish a copy number of original DNA molecules. In addition, the 6 to 8 random bases may be same or different, which are not specifically limited in embodiments of the present disclosure.

In some embodiments, as shown in FIG. 4B, there is one UMI. The UMI 23 is located on the fifth nucleotide single strand 21.

It will be noted that, the UMI 23 may also be located on the sixth nucleotide single strand 22.

In some embodiments, as shown in FIG. 4B, the fifth nucleotide single strand 21 is a forward strand (e.g., the strand disposed from a 5′end to a 3 end in FIG. 4B), and the sixth nucleotide single strand 22 is a reverse strand (e.g., the strand disposed from a 3′ end to a 5′ end in FIG. 4B). The fifth nucleotide single strand 21 includes a sequencing primer sequence 24 and an amplification primer sequence 25. The UMI 23 located on the fifth nucleotide single strand 21 is located between the sequencing primer sequence 24 and the amplification primer sequence 25. The sequencing primer sequence 24 is combined with bases of the sixth nucleotide single strand 22 by complementary base pairing.

It will be noted that, as shown in FIG. 4B, a direction from the 5′end to the 3′ end of the fifth nucleotide single strand 21 is referred to as the first direction X, and the 6 random bases on the UMI 23 are arranged at a 27th base location to a 32th base location. In a subsequent amplification, an amplification primer needs to be complementarily paired at a 1st base location to a 16the base location.

Some embodiments of the present disclosure provide an adapter ligation reagent. The adapter ligation reagent includes first sub-adapters 100 and/or second sub-adapters 110 and/or third sub-adapters 200. In addition, the adapter ligation reagent further includes a T4 DNA ligase, a T4 polynucleotide kinase (T4 PNK), 2× Taq DNA Master Mix, 10×T4 DNA ligase buffer, a polyethylene glycol (PEG), etc. The T4 DNA ligase and the T4 polynucleotide kinase are used for promoting a plurality of adapters (first adapters 10 and/or second adapters 20) to be ligated to DNA single strands. The 2× Taq DNA Master Mix, the 10×T4 DNA ligase buffer and the polyethylene glycol are used for providing a stable pH environment for an adapter ligation reaction. In addition, the polyethylene glycol may include at least one of a polyethylene glycol 4000, a polyethylene glycol 6000 and a polyethylene glycol 8000, which is not specifically limited in embodiments of the present disclosure. The polyethylene glycol 4000 refers to a polyethylene glycol with a molecular weight of 4000. The polyethylene glycol 6000 refers to a polyethylene glycol with a molecular weight of 6000. The polyethylene glycol 8000 refers to a polyethylene glycol with a molecular weight of 8000.

It will be noted that, the 2× Taq DNA Master Mix is a PCR Master Mix, contains a Taq DNA polymerase, dNTPs, a standard Taq enzyme reaction buffer, an enzyme stabilizer and a bromophenol blue dye, and is applicable to conventional PCR applications. A PCR reaction may be performed by simply adding a template and a primer to the product solution during use, which greatly simplifies an operation process and reduces contamination in a PCR operation process. In addition, main components of the 2× Taq DNA Master Mix include 0.1 U/μL of Taq DNA polymerase, 2×PCR reaction buffer solution, 3 mmol/L of magnesium chloride and 0.4 mmol/L of dNTPs. Concentrations of the components may be selected depending on actual needs. In addition, the 2× Taq DNA Master Mix is an existing product, and may be directly purchased commercially. Embodiments of the present disclosure are not limited thereto.

Some embodiments of the present disclosure provide a kit. The kit includes the adapter ligation reagent.

It will be noted that, the kit may be an adapter ligation kit. A kit refers to a box used for containing chemical reagents for detection of chemical components, drug residues, virus types, etc. Here, the kit refers to a box containing the adapter ligation reagent.

Beneficial technical effects of the kit provided in the embodiments of the present disclosure are same as the beneficial technical effects of the adapter provided in the embodiments of the present disclosure, which will not be repeated here.

Some embodiments of the present disclosure provide an application of the UMI 23 in gene sequencing. The UMI includes the at least one random base. Each random base is any one of an A base, a C base, a G base and a T base.

In some embodiments, the gene may include a deoxyribonucleic acid (DNA) molecule or a ribonucleic acid (RNA) molecule for expressing genetic information. UMIs are configured to label different DNA molecules or RNA molecules.

For example, the gene may include ctDNA. The UMIs 23 may be used in UMI adapters to label different ctDNA molecules.

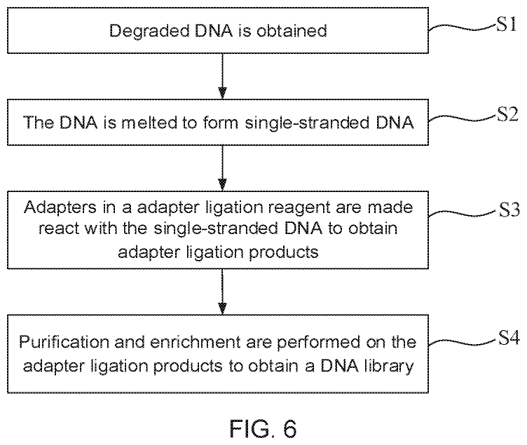

Some embodiments of the present disclosure provide a method for constructing a DNA library or a RNA library. As shown in FIG. 6, the method includes steps S1 to S4.

In S1, degraded DNA is obtained.

For example, the DNA here is fragmented DNA treated by sulfite or DNA that has been highly degraded. Embodiments of the present disclosure are not limited thereto.

In S2, the DNA is melted to form single-stranded DNA.

For example, amplification and incubation are performed by means of a PCR instrument, so that the DNA is melted to obtain the single-stranded DNA. In addition, for some severely degraded DNA samples, single-stranded DNA is present therein. Alternatively, the single-stranded DNA may be obtained commercially. In addition, the single-stranded DNA may be obtained by reverse transcription of messenger RNA (mRNA).

In S3, treatment is performed by using the adapter ligation reagent as described above, so as to make the adapters in the adapter ligation reagent react with the single-stranded DNA to obtain adapter ligation products.

The above adapter ligation reagent including a plurality of adapters (the first sub-adapters, the second adapters and the third sub-adapters) is used to undergo a ligation reaction with the single-stranded DNA to obtain the adapter ligation products.

In S4, purification and enrichment are performed on the adapter ligation products to obtain the DNA library.

Fox example, the purification and the enrichment are performed by adding magnetic beads to the adapter ligation products to obtain the DNA library.

Some embodiments of the present disclosure provide a gene sequencing method. The method includes: performing gene sequencing on DNA or RNA by using the DNA library or the RNA library obtained by the above method for constructing the DNA library or the RNA library.

In the embodiments of the present disclosure, the gene sequencing of the DNA or the RNA is performed by using the DNA library or the RNA library obtained by the above method for constructing the DNA library or the RNA library. Since all DNA molecules or RNA molecules in the constructed DNA or RNA library are ligated with adapters (the first sub-adapters 100, the second sub-adapters 110 and the third sub-adapters 200), and the first sub-adapters 100 increase a percentage of A bases, library construction efficiency is improved by the first sub-adapters 100 and the second sub-adapters 110. In addition, the third sub-adapters 200 include UMIs 23, so that the DNA molecules or the RNA molecules may be labeled by the UMIs 23, which may correct errors generated during sequencing or amplification of a subsequent sequencing process. Therefore, introduction of false positive mutations may be reduced, and the detection accuracy is improved.

In order to objectively evaluate technical effects of the embodiments of the present disclosure, detailed exemplary description of embodiments of the present application is given through the following implementation examples and comparative examples.

In some embodiments of the present disclosure, illustration is made by taking an example where a sequence of the first nucleotide single strand 11 is same as a sequence of the third nucleotide single strand 14, and the sequence thereof is as shown in the following SEQ ID NO: 1.

5′-Phos-AGATCGGAAGAGCGTCGTGTAGGGAAAGA-Spac-3′ SEQ ID NO: 1

Since bases of the first nucleotide single strand 11 are complementarily paired with bases of the second nucleotide single strand 12, and bases of the third nucleotide single strand 14 are complementarily paired with bases of the fourth nucleotide single strand 15, a sequence of the second nucleotide single strand 12 is same as a sequence of the fourth nucleotide single strand 15, and the sequence thereof is as shown in the following SEQ ID NO: 2.

5′-TCTTTCCCTACACGACGCTCTTCCGATCT-3′ SEQ ID NO: 2.

For convenience of explanation in some embodiments of the present disclosure, the first nucleotide single strand 11 is named as a first strand, a structure obtained by ligating the second nucleotide single strand segment 16 (with a sequence of NNNN) to the end of the fourth nucleotide single strand 15 is named as a second strand, a structure obtained by ligating the second nucleotide single strand 12 with a first nucleotide single strand segment 13 (with a sequence of NNNA) is named as a third strand, a structure obtained by ligating the second nucleotide single strand 12 with a first nucleotide single strand segment 13 (with a sequence of NNAN) is named as a fourth strand, a structure obtained by ligating the second nucleotide single strand 12 with a first nucleotide single strand segment 13 (with a sequence of NANN) is named as a fifth strand, and a structure obtained by ligating the second nucleotide single strand 12 with a first nucleotide single strand segment 13 (with a sequence of ANNN) is named as a sixth strand.

The fifth nucleotide single strand 21 and the UMI 23 as a whole is named as a seventh strand, and the sixth nucleotide single strand 22 is named as an eighth strand. Sequences of the first strand to the eight strand are as shown in Table 1 below.

TABLE 1

| | | Sequence |
|---|---|---|
| Number | Sequence | number |
| First strand | 5′-Phos- | SEQ ID NO: 3 |
| | AGATCGGAAGAGCGTCGTGTAGGGAAAGA- | |
| | Spac-3′ | |
| Second strand | 5′- | SEQ ID NO: 4 |
| | TCTTTCCCTACACGACGCTCTTCCGATCTN*N* | |
| | N*N-3′ | |
| Third strand | 5′- | SEQ ID NO: 5 |
| | TCTTTCCCTACACGACGCTCTTCCGATCTN*N* | |
| | N*A-3′ | |
| Fourth strand | 5′- | SEQ ID NO: 6 |
| | TCTTTCCCTACACGACGCTCTTCCGATCTN*N* | |
| | A*N-3′ | |
| Fifth strand | 5′- | SEQ ID NO: 7 |
| | TCTTTCCCTACACGACGCTCTTCCGATCTN*A* | |
| | N*N-3′ | |
| Sixth strand | 5′- | SEQ ID NO: 8 |
| | TCTTTCCCTACACGACGCTCTTCCGATCTA*N* | |
| | N*N-3′ | |
| Seventh strand | 5′- | SEQ ID NO: 9 |
| | AGTTCAGACGTGTGCTCTTCCGATCTNNNNN | |
| | NAGATCGGAAG*T-3′ | |
| Eighth strand | 3′-TCTAGCCTTC-Phos-5′ | SEQ ID NO: 10 |

As can be seen from Table 1 above, the sequences of the first nucleotide single strand segments 13 include NNNA, NNAN, NANN and ANNN, and the sequence of the second nucleotide single strand segment 16 includes NNNN, where N represents a random base, and N is any one of an A base, a C base, a G base and a T base. * represents a thio modification which ensures that the DNA is not degraded. The “Phos” represents a phosphate group modification, and the “Spac” represents a C3 spacer.

### 1. Adapter Synthesis

### Adapter Synthesis Implementation Example

In a step 1, the first strand to the eighth strand are each resuspended to obtained solutions with a concentration of 100 μM and a volume of 100 μL.

In a step 2, a buffer solution reagent with a volume of 100 μL is prepared. The reagent is composed of:
10 mM of trihydroxymethyl aminomethane (Tris)-HCl buffer at pH 7.5, 2 mM of EDTA and 50 mM of NaCl.

In a step 3, 10 μL of a solution of the first strand and 10 μL of a solution of the second strand are taken and placed into a PCR tube labeled with “Adapter 1-1”, 10 μL of the solution of the first strand and 10 μL of a solution of the third strand are taken and placed into a PCR tube labeled with “Adapter 1-2”, 10 μL of the solution of the first strand and 10 μL of a solution of the fourth strand are taken and placed into a PCR tube labeled with “Adapter 1-3”, 10 μL of the solution of the first strand and 10 μL of a solution of the fifth strand are taken and placed into a PCR tube labeled with “Adapter 1-4”, 10 μL of the solution of the first strand and 10 μL of a solution of the sixth strand are taken and placed into a PCR tube labeled with “Adapter 1-5”, and 10 μL of a solution of the seventh strand and 10 μL of a solution of the eighth stand are taken and placed into a PCR tube labeled with “Adapter 2”; 80 μL of the buffer solution is added into each of the PCR tubes; fully and uniformly mixing is performed; and centrifugation is performed for 10 s.

In a step 4, all of the PCR tubes are placed in a PCR instrument for denaturation for 10 min at 95° C.

In a step 5, after reactions are finished, the PCR instrument is directly turned off; and the PCR tubes are taken out after a temperature is reduced to a room temperature.

In a step 6, 1 μL of products in each PCR tube is taken to carry out substance detection in a full-automatic nucleic acid fragment analyzer (Qsep 100), and adapters (first sub-adapters 100, second sub-adapters 110 and third sub-adapters 200) as shown in FIGS. 2A to 2D, 3B and 4B are obtained.

### 2. Library Construction and Sequencing:

### Implementation Example 1

In a step 1, cell-free DNA (cfDNA) standards with a plurality of mutation sites and a mutation frequency of 1% customized from GeneWell are used as samples. Library construction may be performed directly by using the cfDNA standards as samples.

In a step 2, 1 ng to 200 ng (e.g., 1 ng, 5 ng, 10 ng, 50 ng or 200 ng) of fragmented DNA treated by sulfite (i.e., the cfDNA standards) is added into a PCR tube, and diluted to a total volume of 30 μL by adding ultrapure water.

In a step 3, the PCR tube in the step 2 is placed into the PCR instrument for incubation for 5 min at 95° C.; and then the PCR tube is cooled below 0′C and left to stand for 2 min so as to fully melt the DNA into single-stranded DNA.

In a step 4, reagents in Table 2 are thawed and uniformly mixed, and then sequentially added into the PCR tube in the step 3 at a temperature below 0° C.; the mixture is lightly blown and sucked by using a pipette or shaken so as to mix well; and then instant centrifugation is performed to make a reaction liquid reach a bottom of the tube. Adapters here are the adapters synthesized in Adapter Synthesis Implementation Example. The adapters are as shown in FIGS. 2A to 2D (the first sub-adapters 100) and FIG. 3B (the second sub-adapters 110), and each kind of sub-adapters is equal in number.

TABLE 2

| | | |
|---|---|---|
| Reagent name | Concentration | Volume |

| | | | |
|---|---|---|---|
| DNA (i.e., the single | 10 | U/μL | 30 μL |

| | | |
|---|---|---|
| stranded DNA in the step | | |
| 3) | | |
| T4 DNA ligase | — | 2 μL |
| 10X T4 DNA ligation | 50% | 4 μL |
| buffer | | |
| PEG 4000 | | 2 μL |

| | | | |
|---|---|---|---|
| Adapter | 20 | μM | 2 μL |

In a step 5, the PCR tube in the step 4 is placed in the PCR instrument for reaction for 30 min at 20′C and then denaturation for 2 min at 95° C.

In a step 6, 40 μL of products in the PCR tube in the step 5 are taken and placed into a new PCR tube at a temperature below 0′C; 40 μL of 2× Taq DNA Master Mix and 3 μL of primers with a concentration of 10 μM are added therein; the mixture is lightly blown and sucked by a pipette or shaken so as to mix well, and then instant centrifugation is performed to make a reaction liquid reach a bottom of the tube.

In a step 7, the PCR tube in the step 6 is placed in the PCR instrument for denaturation for 2 min at 98° C., annealing for 2 min at 60° C., extension for 5 min at 70° C. and preservation at 4° C. to obtain adapter ligation products.

In a step 8, the ligation products are purified as follows. Magnetic beads with a volume 1.2 times a volume of the adapter ligation products into the adapter ligation products; the mixture is fully and uniformly mixed, left to stand for 5 min at the room temperature, and placed on a magnetic frame to enable the magnetic beads to perform complete adsorption and enable the solution to be clarified; a supernatant is carefully removed; 200 μL of 80% ethanol is added for rinsing, incubation is performed for 30 s to 60 s at the room temperature, a supernatant is carefully removed; the previous operation is repeated once; after the magnetic beads are dried, 31 μL of ultrapure water is added for elution, the mixture is left to stand for 3 min at the room temperature, and then placed on the magnetic frame; and after the solution is clear, and 30 μL of a supernatant is absorbed for later use, so that purified products are obtained.

In a step 9, reagents in Table 3 are thawed, and then mixed well, and then placed at a temperature of below 0′C; 30 μL of the purified products obtained in the step 8 is taken and placed in a new PCR tube, and the reagents in Table 3 are sequentially added therein; the mixture is gently blown and sucked by using a pipettor or shaken so as to mix well; and then instant centrifugation is performed to make a reaction liquid reach a bottom of the tube. Adapters in Table 3 are adapters (the third sub-adapters 200) as shown in FIG. 4B.

TABLE 3

| Reagent Name | Concentration | Volume |
|---|---|---|
| T4 DNA ligase | — | 5 μL |
| 10X T4 DNA ligation | 50% | 5 μL |
| buffer | | |
| PEG 4000 | | 5 μL |
| Adapter | 20 μM | 5 μL |

In a step 10, the PCR tube in the step 9 is placed into the PCR instrument to perform ligation reaction for 15 min at 20° C., and then preserved at 4° C.

In a step 11, products are enriched and purified as follows. Magnetic beads with a volume one times a volume of ligation products obtained in the step 10 into the amplification products; the mixture is fully and uniformly mixed, and then left to stand for 5 min at the room temperature, and placed on the magnetic frame to enable the magnetic beads to perform complete adsorption and enable the solution to be clarified; a supernatant is carefully removed; 200 μL of 80% ethanol is added for rinsing, incubation is performed for 30 s to 60 s at the room temperature, a supernatant is carefully removed; the previous operation is repeated once; after the magnetic beads are dried, 22 μL of ultrapure water is added for elution, the mixture is left to stand for 3 min at the room temperature, and then placed on the magnetic frame; and after the solution is clear, 20 μL of a supernatant is absorbed and placed into a new PCR tube.

In a step 12, 20 μL of products in the step 11 are taken and placed into a new PCR tube; 25 μL of 2×HIFI Uracil PCR Mix and 5 μL of primer Mix are added therein, the mixture is gently blown and sucked by using a pipette or shaken so as to mix well, and then instant centrifugation is performed to make a reaction liquid reach a bottom of the tube.

The primer Mix includes two kinds of primers, which are generally divided into i5 primers and i7 primers in an illiminina sequencing platform. In addition, an i5 primer includes an i5 Index, and an i7 primer includes an i7 Index. Specific sequences of the i5 primer and the i7 primer are as shown in Table 4 below.

TABLE 4

| | | |
|---|---|---|
| i5 primer | 5′-AATGATACGGCGACCACCGAGATCTAC | SEQ ID |
| | ACIndexACACTTTCCCTACACGACGCTCT | NO: 11 |
| | TCCGATCT-3′ | |
| i7 primer | 5′-CAAGCAGAAGACGGCATACGAGATInd | SEQ ID |
| | exGTGACTGGAGAGTTCAGACGTGTGCTCT | NO: 12 |
| | TCCGATCT-3′ | |

In a step 13, the PCR tube in the step 12 is placed in the PCR instrument for pre-denaturation for 1 min at 98° C., and then cyclic reaction is performed 5 to 10 times, the cyclic reaction including denaturation for 20 s at 98′C, primer annealing for 30 s at 60° C., and product extension for 30 s at 72° C.; and after the cyclic reaction is completed, final extension is performed for 3 min at 72° C., and finally temporary preservation is carried out at 4° C.

In a step 14, library concentration is determined. 1 μL of products in the step 13 is taken to be detected by using Qubit 4.0 Fluorometer.

Step 15, sequencing on a computer is performed. A Novaseq 6000 (Illumina) instrument is used for sequencing on the computer, and the FastQC software is used for analyzing basic quality control of offline data. Actual detected sites and mutations are substantially consistent with theoretical values, and specific detection results are as shown in Table 5 and Table 6 below.

In addition, since a single library corresponds to a single sample DNA (the DNA in the step 2), and the final step in the process of constructing a library is Index primer amplification, each sample is given an added Index (including an i5Index and an i7 Index) after the primer amplification is completed, and the set of the i5Index and the i7 Index determines information of the sample. Thus, in order to facilitate mixing of a plurality of samples DNA in a sequencing reaction, Index primer amplification is performed after the library construction process of each sample DNA. That is, each sample DNA is labeled so as to facilitate identification during sequencing. Different sequencing instruments correspond to different Index sequences. Indexes corresponding to each sequencing instrument include 16 sequences. The Index sequences are specifically shown in Tables 7 to 9 below.

TABLE 7
i5 Index (Novaseq 6000 v1.0, Hiseq2000/2500,
Miseq)

| Index sequence number | 5′-3′ |
|---|---|
| SEQ ID NO: 13 | CGACCATT |
| SEQ ID NO: 14 | GATAGCGA |
| SEQ ID NO: 15 | AATGGACG |
| SEQ ID NO: 16 | CGCTAGTA |
| SEQ ID NO: 17 | TCTCTAGG |
| SEQ ID NO: 18 | ACATTGCG |
| SEQ ID NO: 19 | TGAGGTGT |
| SEQ ID NO: 20 | AATGCCTC |
| SEQ ID NO: 21 | CTGGAGTA |
| SEQ ID NO: 22 | GTATGCTG |
| SEQ ID NO: 23 | TGGAGAGT |
| SEQ ID NO: 24 | CGATAGAG |
| SEQ ID NO: 25 | CTCATTGC |
| SEQ ID NO: 26 | ACCAGCTT |
| SEQ ID NO: 27 | GAATCGTG |
| SEQ ID NO: 28 | AGGCTTCT |

TABLE 8
i5 Index (Novaseq 6000 v1.5, Hiseq 3000/4000,
Miniseq)

| Index sequence number | 5′-3′ |
|---|---|
| SEQ ID NO: 29 | AATGGTCG |
| SEQ ID NO: 30 | TCGCTATC |
| SEQ ID NO: 31 | CGTCCATT |
| SEQ ID NO: 32 | TACTAGCG |
| SEQ ID NO: 33 | CCTAGAGA |
| SEQ ID NO: 34 | CGCAATGT |
| SEQ ID NO: 35 | ACACCTCA |
| SEQ ID NO: 36 | GAGGCATT |
| SEQ ID NO: 37 | TACTCCAG |
| SEQ ID NO: 38 | CAGCATAC |
| SEQ ID NO: 39 | ACTCTCCA |
| SEQ ID NO: 40 | CTCTATCG |
| SEQ ID NO: 41 | GCAATGAG |
| SEQ ID NO: 42 | AAGCTGGT |
| SEQ ID NO: 43 | CACGATTC |
| SEQ ID NO: 44 | AGAAGCCT |

TABLE 9
i7 index (all illuminas systems)

| Index sequence number | 5′-3′ |
|---|---|
| SEQ ID NO: 45 | GCCTATCA |
| SEQ ID NO: 46 | CTTGGATG |
| SEQ ID NO: 47 | AGTCTCAC |
| SEQ ID NO: 48 | CTCATCAG |
| SEQ ID NO: 49 | TGTACCGT |
| SEQ ID NO: 50 | AAGTCGAG |
| SEQ ID NO: 51 | CACGTTGT |
| SEQ ID NO: 52 | TCACAGCA |
| SEQ ID NO: 53 | CTACTTGG |
| SEQ ID NO: 54 | CCTCAGTT |
| SEQ ID NO: 55 | TCCTACCT |
| SEQ ID NO: 56 | ATGGCGAA |
| SEQ ID NO: 57 | CTTACCTG |
| SEQ ID NO: 58 | CTCGATAC |
| SEQ ID NO: 59 | TCCGTGAA |
| SEQ ID NO: 60 | TAGAGCTC |

### Implementation Example 2

Steps in Implementation Example 2 are substantially same as those in Implementation Example 1, which will not be repeated here. A difference is that, in a step 4, a library is constructed by using the adapters which are as shown in FIGS. 2A, 2B, 3B and 4B and synthesized in the Adapter Synthesis Implementation Example. Each kind of sub-adapters is equal in number. Actual detected sites and mutations are substantially consistent with theoretical values, and specific detection results are as shown in Table 5 and Table 6 below.

### Comparative Example

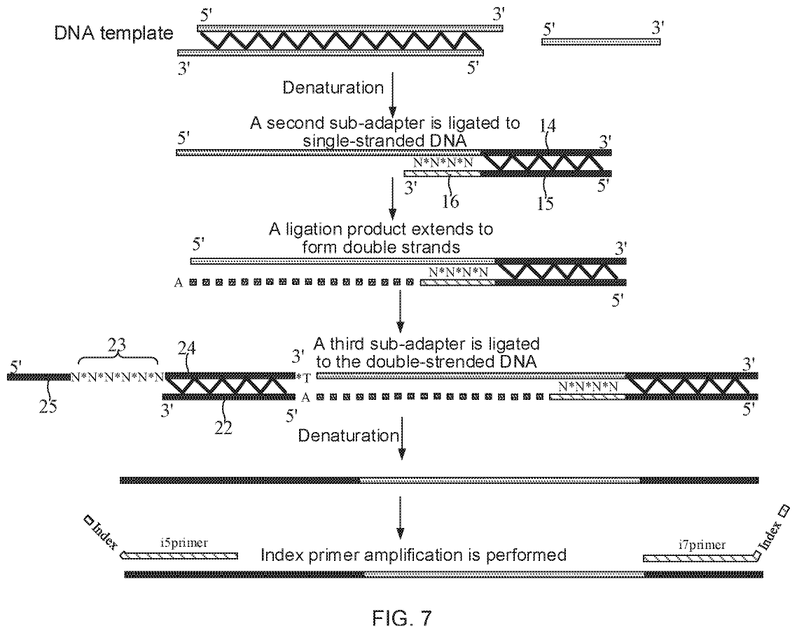

Steps in Comparative Example are substantially same as those in Implementation Example 1, which will not be repeated here. A difference is that, in a step 4, a library is constructed by using the adapters which are as shown in FIGS. 3B and 4B and synthesized in the Adapter Synthesis Implementation Example. Each kind of sub-adapters is equal in number. A library construction process in this example is as shown in FIG. 7. In addition, similarly, library construction processes of Implementation Example 1 and Implementation Example 2 may refer to FIG. 7, which will not be detailed here. Although actual detected sites and mutations in this example are substantially consistent with theoretical values, but are inferior to those in Implementation Example 1 and Implementation Example 2. Specific detection results are as shown in Table 5 and Table 6 below.

TABLE 5

| Implementation | Sample | DNA addition | Number of | Library yield |
|---|---|---|---|---|
| Example | number | amount (ng) | PCR cycles | (ng) |
| Implementation | 1 | 1 | 14 | 2100 |
| Example 1 | | | | |
| Implementation | 2 | 1 | 14 | 2060 |
| Example 1 | | | | |
| Implementation | 3 | 5 | 12 | 1980 |
| Example 1 | | | | |
| Implementation | 4 | 5 | 12 | 1990 |
| Example 1 | | | | |
| Implementation | 5 | 10 | 11 | 2050 |
| Example 1 | | | | |
| Implementation | 6 | 10 | 11 | 2030 |
| Example 1 | | | | |
| Implementation | 7 | 50 | 9 | 1980 |
| Example 1 | | | | |
| Implementation | 8 | 50 | 9 | 1996 |
| Example 1 | | | | |
| Implementation | 9 | 200 | 6 | 1890 |
| Example 1 | | | | |
| Implementation | 10 | 200 | 6 | 1900 |
| Example 1 | | | | |
| Implementation | 1 | 1 | 14 | 1600 |
| Example 2 | | | | |
| Implementation | 2 | 1 | 14 | 1580 |
| Example 2 | | | | |
| Implementation | 3 | 5 | 12 | 1320 |
| Example 2 | | | | |
| Implementation | 4 | 5 | 12 | 1310 |
| Example 2 | | | | |
| Implementation | 5 | 10 | 11 | 1360 |
| Example 2 | | | | |
| Implementation | 6 | 10 | 11 | 1380 |
| Example 2 | | | | |
| Implementation | 7 | 50 | 9 | 1180 |
| Example 2 | | | | |
| Implementation | 8 | 50 | 9 | 1205 |
| Example 2 | | | | |
| Implementation | 9 | 200 | 6 | 1070 |
| Example 2 | | | | |
| Implementation | 10 | 200 | 6 | 1110 |
| Example 2 | | | | |
| Comparative | 1 | 1 | 14 | 1400 |
| Example | | | | |
| Comparative | 2 | 1 | 14 | 1350 |
| Example | | | | |
| Comparative | 3 | 5 | 12 | 1250 |
| Example | | | | |
| Comparative | 4 | 5 | 12 | 1210 |
| Example | | | | |
| Comparative | 5 | 10 | 11 | 1200 |
| Example | | | | |
| Comparative | 6 | 10 | 11 | 1125 |
| Example | | | | |
| Comparative | 7 | 50 | 9 | 1000 |
| Example | | | | |
| Comparative | 8 | 50 | 9 | 1050 |
| Example | | | | |
| Comparative | 9 | 200 | 6 | 950 |
| Example | | | | |
| Comparative | 10 | 200 | 6 | 980 |
| Example | | | | |

Generally, in a library construction process, ligation efficiency of a library may be evaluated by absolute quantification of ligation products through fluorescent quantitative PCR. Since PCR amplification is performed after a ligation reaction is completed, it may also be possible to perform the evaluation by library yield comparison under a condition of the same DNA addition amount and the same number of amplification cycles. In embodiments of the present disclosure, library yields are used for quantification evaluation of ligation efficiency. As can be seen from the experimental data in Table 5, an average of library yields in Implementation Example 1 is about 2000 ng, an average of library yields in Implementation Example 2 is about 1300 ng, and an average of library yields in Comparison Example is about 1100 ng. Both Implementation Example 1 and Implementation Example 2 are superior to those of the Comparison Example in library yield. Thus, it is demonstrated that the adapters in Implementation Example 1 and Implementation Example 2 improve efficiency of complementary pairing with the single-stranded DNA, thereby improving ligation efficiency and ultimately improving the library yield.

TABLE 6

| | | | Theoretical | Actually detected |
|---|---|---|---|---|
| Implementation | | | mutation | mutation |
| Example | Gene | Mutation site | frequency | frequency |
| Implementation | EGFR | T790M | 1% | 1.10% |
| Example 1 | EGFR | L858R | 1% | 1.11% |
| | KRAS | G12D | 1% | 0.95% |
| | NRAS | Q61K | 1% | 0.94% |
| Implementation | EGFR | T790M | 1% | 1.05% |
| Example 2 | KRAS | G12D | 1% | 0.91% |
| | KRAS | G13D | 1% | 1.10% |
| | NRAS | Q61K | 1% | 0.90% |
| Comparative | EGFR | T790M | 1% | 1.15% |
| Example | KRAS | G12D | 1% | 0.95% |
| | KRAS | G13D | 1% | 0.98% |
| | NRAS | Q61K | 1% | 0.93% |

In Table 6 above, the actually detected mutation frequencies of different mutation sites of the selected gene in Implementation Example 1 are substantially between 0.94% to 1.11%, which are more accurate compared with the theoretical mutation frequency (1%); the actually detected mutation frequencies of the different mutation sites of the selected gene in Implementation Example 2 are substantially between 0.90% to 1.10%, which are also more accurate compared with the theoretical mutation frequency; and the actually detected mutation frequencies of the different mutation sites of the selected gene in Comparative Example are substantially between 0.93% to 1.15%, which are also more accurate compared with the theoretical mutation frequency. However, Comparative Example fluctuates more compared to Implementation Example 1 and Implementation Example 2.

To sum up, by using the adapters and the UMIs in the embodiments of the present application, it may be possible to ensure diversity of adapters, so that different original DNA fragments may be labeled, and the library yield is improved; and moreover, noise mutations introduced by PCR amplification or sequencing may be eliminated to a certain extent, which improves detection accuracy.

The foregoing descriptions are merely specific implementations of the present disclosure. However, the protection scope of the present disclosure is not limited thereto. Changes or replacements that any person skilled in the art could conceive of within the technical scope of the present disclosure shall be included in the protection scope of the present disclosure. Therefore, the protection scope of the present disclosure shall be subject to the protection scope of the claims.

1. An adapter comprising:
at least one first sub-adapter, wherein each first sub-adapter includes:
a first nucleotide single strand and a second nucleotide single strand, the first nucleotide single strand being complementarily paired with the second nucleotide single strand; and
a first nucleotide single strand segment, the first nucleotide single strand segment being ligated to an end of the first nucleotide single strand or an end of the second nucleotide single strand, the first nucleotide single strand segment including at least one random base and at least one adenine (A) base, and each random base being any one of an A base, a cytosine (C) base, a guanine (G) base and a thymine (T) base.

2. The adapters according to claim 1, wherein the first nucleotide single strand segment includes a plurality of random bases and at least one A base, and the plurality of random bases are arranged consecutively; or
the first nucleotide single strand segment includes a plurality of A bases and at least one random base, and the plurality of A bases are arranged consecutively.

3. The adapters according to claim 1, wherein the first nucleotide single strand segment includes a plurality of random bases and at least one A base, and one or more A bases of the at least one A base are disposed between two random bases of the plurality of random bases; or
the first nucleotide single strand segment includes a plurality of A bases and at least one random base, and one or more random bases of the at least one random base are disposed between two A bases of the plurality of A bases.

4. The adapter according to claim 1, wherein the first nucleotide single strand segment includes three random bases and one A base.

5. The adapter according to claim 1, wherein the adapter comprises a plurality of first sub-adapters; and among the plurality of first sub-adapters, at least two first sub-adapters are different in that random bases and A bases of respective first nucleotide single strand segments are arranged in different orders.

6. The adapters according to claim 5, wherein the adapter comprises four first sub-adapters, and four first nucleotide single strand segments of the four first sub-adapters are different in that random bases and A bases of respective first nucleotide single strand segments are arranged in different orders.

7. An adapter, comprising:
at least one second sub-adapter, wherein each second sub-adapter includes:
a third nucleotide single strand and a fourth nucleotide single strand, the third nucleotide single strand being complementarily paired with the fourth nucleotide single strand; and
a second nucleotide single strand segment, the second nucleotide single strand segment being ligated to an end of the third nucleotide single strand or an end of the fourth nucleotide single strand, the second nucleotide single strand segment including at least one random base, each random base being any one of an A base, a C base, a G base and a T base.

8. The adapters according to claim 7, wherein the second nucleotide single stranded segment includes four random bases.

9. An adapter ligation reagent, comprising:
the adapter according to claim 1.

10. A kit, comprising:
the adapter ligation reagent according to claim 9.

11. The kit according to claim 10, further comprising:
a third sub-adapter, wherein the third sub-adapter includes:
a fifth nucleotide single strand and a sixth nucleotide single strand, the fifth nucleotide single strand being complementarily paired with the sixth nucleotide single strand; and
at least one unique molecular identifier (UMI), each UMI being located on the fifth nucleotide single strand or the sixth nucleotide single strand.

12. The kit according to claim 11, wherein the UMI includes:
at least one random base, each random base being any one of an A base, a C base, a G base and a T base.

13. The kit according to claim 12, wherein the at least one random base includes at least six random bases.

14. The kit according to claim 11, wherein the at least one UMI includes one UMI, and the UMI is located on the fifth nucleotide single strand.

15. The kit according to claim 14, wherein the fifth nucleotide single strand is a forward strand, and the sixth nucleotide single strand is a reverse strand; and
the fifth nucleotide single strand includes a sequencing primer sequence and an amplification primer sequence, the UMI located on the fifth nucleotide single strand is located between the sequencing primer sequence and the amplification primer sequence, and the sequencing primer sequence is combined with bases of the sixth nucleotide single strand through complementary base pairing.

16. A method for constructing a deoxyribonucleic acid (DNA) library, comprising:
obtaining degraded DNA;
melting the degraded DNA to form single-stranded DNA;
performing treatment, by using the adapter ligation reagent according to claim 18, to make the at least one first sub-adapter and the at least one second sub-adapter of the adapter ligation reagent react with the single-stranded DNA to obtain adapter ligation products; and
purifying and enriching the adapter ligation products to obtain the DNA library.

17. A method for sequencing a gene, comprising:
performing gene sequencing on DNA obtained by using the method for constructing the DNA library according to claim 19.

18. The adapter ligation reagent according to claim 9, further comprising at least one second sub-adapter, wherein each second sub-adapter includes:
a third nucleotide single strand and a fourth nucleotide single strand, the third nucleotide single strand being complementarily paired with the fourth nucleotide single strand; and
a second nucleotide single strand segment, the second nucleotide single strand segment being ligated to an end of the third nucleotide single strand or an end of the fourth nucleotide single strand, the second nucleotide single strand segment including at least one random base, each random base being any one of an A base, a C base, a G base and a T base.

19. The adapter ligation reagent according to claim 18, further comprising a third sub-adapter, wherein the third sub-adapter includes:
a fifth nucleotide single strand and a sixth nucleotide single strand, the fifth nucleotide single strand being complementarily paired with the sixth nucleotide single strand; and
at least one unique molecular identifier (UMI), each UMI being located on the fifth nucleotide single strand or the sixth nucleotide single strand.

20. The method according to claim 16, wherein the adapter ligation reagent further includes a third sub-adapter, the third sub-adapter includes:
a fifth nucleotide single strand and a sixth nucleotide single strand, the fifth nucleotide single strand being complementarily paired with the sixth nucleotide single strand; and
at least one unique molecular identifier (UMI), each UMI being located on the fifth nucleotide single strand or the sixth nucleotide single strand; and
the method comprises:
performing treatment, by using the adapter ligation reagent, to make the at least one first sub-adapter, the at least one second sub-adapter and the third sub-adapter of the adapter ligation reagent react with the single-stranded DNA to obtain the adapter ligation products.
